(19) United States
(12) Patent Application Publication
(10) Pub. No.: US 2024/0206959 A1
(43) Pub. Date: Jun. 27, 2024

(54) SYSTEMS AND METHODS FOR SELECTIVELY INDUCING VASOCONSTRICTION TO INCREASE THERAPEUTIC UPTAKE IN TISSUE

(71) Applicant: TriSalus Life Sciences, Inc., Westminster, CO (US)

(72) Inventors: Erik Dean Olson, Castle Rock, CO (US); Michael Brick Markham, Boulder, CO (US); David Benjamin Jaroch, Arvada, CO (US); Mikael Trollsas, San Jose, CA (US)

(73) Assignee: TriSalus Life Sciences, Inc., Westminster, CO (US)

(21) Appl. No.: 18/087,508

(22) Filed: Dec. 22, 2022

(51) Int. Cl. A61B 18/14; A61B 8/12

(52) U.S. Cl. CPC A61B 18/1492; A61B 8/12; A61B 2018/00577

(57) ABSTRACT

A microcatheter is provided with a vascular spasm-inducing probe at its distal end. The spasm-inducing probe can include an electrical current, radiofrequency energy, ultrasound, chemical, or thermal modulator. When the spasm-inducing probe is activated in a vessel, the vessel narrows about the element. This permits a therapeutic agent to be delivered through the infusion lumen and out of the orifice under high pressure, while the narrowing of the vessel operates as a pseudo-valve and prevents reflux of the therapeutic agent past the narrowing of the vessel.

### BACKGROUND

### 1. Field of the Invention

The medical methods described herein relate generally to methods for intravenously treating a target organ for cancer or other diseases.

### 2. State of the Art

In some instances, systemic treatments are used to treat disease within a patient. The effectiveness of some such systemic treatments can vary due at least in part to the therapy (e.g., a radio-embolization agent, a biologic agent and/or other treatment formulation) not reaching target tissue. For example, in the treatment of some diseases such as cancer and/or diabetes, it may be desirable to deliver biological cells to an organ where efficient and safe engraftment can be achieved.

The ineffective result of systemic chemotherapy is at least in part due to an insufficient drug concentration within the tumor because of dose-limited toxicity in bone marrow and epithelial tissue. Another complication can be peripheral neuropathy. Yet another obstacle can result from the systemic use of check point inhibitors causing over-activation of the immune system. Since systemic chemotherapy is limited in its effectiveness or can have significant complications, treatments other than systemic chemotherapy can be desirable for many types of cancer patients.

One alternative treatment includes local intra-arterial delivery of chemotherapy. Intra-arterial infusion allows higher drug concentration to reach a tumor. Furthermore, intra-arterial chemotherapy can also take advantage of the first pass effect of chemotherapeutics, generating higher-level drug concentrations at the tumor cell membrane and therefore enhancing cellular drug uptake as compared to intravenous systemic infusion. In addition, local delivery can reduce systemic side effects resulting when the drug fails to remain localized and disperses.

Standard end-hole catheters permit limited control of infused local treatment. Contribution of the infusion adds local volume into a nearly incompressible system. The infusion treatment will flow from an area of high pressure to an area of lower pressure. The added fluid volume of the treatment is forced to move somewhere and, if the downstream resistance and pressure is higher than the upstream resistance, reflux to non-target areas will occur.

In order to alleviate certain of these issues, co-owned U.S. Pat. Nos. 8,696,698, 9,968,840, and 10,588,636 describe various pressure-controlled therapeutic delivery devices in the form of an infusion catheter having an integrated microvalve mounted at the distal end of the catheter. The microvalve dynamically expands and contracts within a blood vessel in relation to the surrounding fluid pressure in the vessel. A treatment can be infused through the catheter. When the treatment agent is infused, the pressure in the vessel downstream (distal) of the treatment at times can become higher than that upstream (proximal) of the treatment, allowing the microvalve to block reflux of the agent. In addition, the microvalve permits infusion into the target tissue at modified pressure, targeting the treatment into the desired tissue.

These pressure-controlled therapeutic devices work extremely well for their intended purposes. However, there may be treatment scenarios in which it would be desirable to advance a treatment device into smaller or more tortuous vessels than can accommodate current microvalves. In addition, current size mechanical microvalves have limits on trackability through these vessels.

### SUMMARY

Systems and methods are provided for the pressure-controlled delivery of a therapeutic. The systems are trackable through vessels feeding tumors and do not rely on mechanical operation of a microvalve. In accord with embodiments, each system includes a microcatheter having a proximal end and a distal end, an infusion lumen extending through the microcatheter from the proximal end to the distal end and opening at a distal orifice. The microcatheter is provided with a vascular spasm-inducing system. For purposes herein, a vasospasm is the narrowing of a vessel caused by constriction of smooth muscle lining the vessel, typically due to irritatation of the vessel. Vasospasm, vasoconstriction and more generally spasm are terms relating to this same phenomena and are used interchangeably herein. The magnitude of vasospasm occurs along a spectrum ranging from mild narrowing of the vessel to complete occlusion. The vascular spasm inducing system includes a spasm-inducing probe at or adjacent the distal end of the microcatheter, and an actuator to trigger the spasm-inducing element. By way of example only, the vascular spasm-inducing probe can be a radiofrequency (RF) emitter, such as of an electric current or voltage or magnetic or electromagnetic field, an ultrasound emitter, a chemical emitter, a thermal probe, and particularly a cryogenic probe, an infrared probe, a mechanical vibratory emitter, or a mechanical irritation device. Vasospasm-inducing systems described herein include devices adapted to causing a constriction of a blood vessel to varying degrees of magnitude.

When the vascular spasm-inducing probe is triggered in a vessel, the induced spasm in the vessel causes a narrowing about the spasm-inducing probe. This permits a therapeutic agent to be delivered through the infusion lumen and out of the orifice a range of pressure determined by the user, while the narrowing of the vessel operates as a pseudo-valve or natural valve and prevents reflux of the therapeutic agent past the narrowing in the vessel.

In one method, the system is used to deliver a therapeutic agent downstream of the narrowing under a pressure determined by the user and is then withdrawn from the patient.

In another method, the system is used to control and modify flow within the vasculature before delivery of the therapeutic agent in order to optimize flow of the agent to the tumor. Before delivery of therapy, the spasm-inducing probe is activated to cause a narrowing and thus reduce flow in a vessel. When flow is reduced, the healthy downstream vessels react by constricting; however, the tumor vessels do not properly react to the lower pressure and remain substantially open. This creates a vascular pathway in which remaining flow is directed toward the tumor. The therapeutic is then infused and is directed to the tumor.

In accord with another method, the system is used to create a constriction leading to a bleeding vessel system to reduce bleeding. Then a therapeutic agent may be delivered to further treat the bleeding vessel.

In accord with another embodiment of a system herein, a microcatheter is provided substantially as described above; however, the spasm-inducing probe is modified to be an ablation probe. In accord with preferred embodiments, the ablation probe can be implemented using the same modalities for inducing spasm; that is, the ablation probe can be an RF emitter, an ultrasound emitter, a chemical emitter, a thermal probe, an infrared emitter, a mechanical vibratory emitter, or an irritation device, but all operated with sufficient energy to cause tissue ablation rather than vascular spasm. In addition, the ablation probe can be other energy emitters that can be operated to ablate vascular tissue, such as a microwave emitter. As described above, the system includes an actuator at the proximal end of the microcatheter, and the actuator is operably connected to the ablation probe. The microcatheter is not necessarily required to infuse a therapeutic agent, as the intended therapeutic effect is provided by ablation of tissue, as described below. However, the microcatheter preferably includes a central lumen sized for receiving a guidewire.

In use, a mapping procedure is performed to identify the significant arterial vessels feeding a solid tumor. Once a vessel is identified, the femoral or radial artery is accessed, and a guidewire is advanced from the femoral or radial artery to a significant vessel feeding the tumor. The microcatheter is then advanced over the guidewire to the target vessel. Then, the guidewire is removed. Alternatively, the microcatheter is advanced directly, without a guidewire. The ablation probe is then activated to ablate the surrounding tumor-feeding arterial vessel. Ablation results in occlusion and/or collapse of the artery, preventing nourishing blood flow to the tumor. This will prevent further growth of the tumor and/or reduce the size of the tumor. The system can be repositioned for ablation in additional vessels or removed.

The systems can be used to provide treatment in vessels wherever an endhole or conventional catheter, a balloon catheter, or microvalve catheter would be used to infuse a therapy into a vessel. In particular, the systems and methods can be used in vessels to treat tumors in organs throughout the human body.

### BRIEF DESCRIPTION OF THE DRAWINGS

FIG. 1 is a schematic illustration of a treatment system described herein.

FIG. 2 is a schematic illustration of a distal end of the treatment system of FIG. 1.

FIG. 3 is a schematic illustration of another embodiment of a distal end of the treatment system of FIG. 1.

FIG. 4 is a schematic illustration of yet another embodiment of a distal end of the treatment system of FIG. 1.

FIG. 5 is a schematic illustration of a further embodiment of a distal end of the treatment system of FIG. 1.

FIGS. 6 through 8 illustrate one method of using the treatment system described herein.

FIGS. 9 and 10 illustrate another method of using the treatment system described herein.

FIG. 11 is a schematic illustration of another treatment system described herein.

FIGS. 12 and 13 illustrate a method of using the treatment system shown in FIG. 11.

FIGS. 14 and 15 are schematic illustrations of a further embodiment of a distal end of the treatment system of FIG. 1.

FIG. 16 illustrates a method of using the treatment systems described herein.

### DETAILED DESCRIPTION OF THE PREFERRED EMBODIMENTS

With reference to the following description, the terms “proximal” and “distal” are defined in reference to the hand of a user of the devices and systems described herein, with the term “proximal” being closer to the user's hand, and the term “distal” being further from the user's hand such as to often be located further within a body of the patient during use.

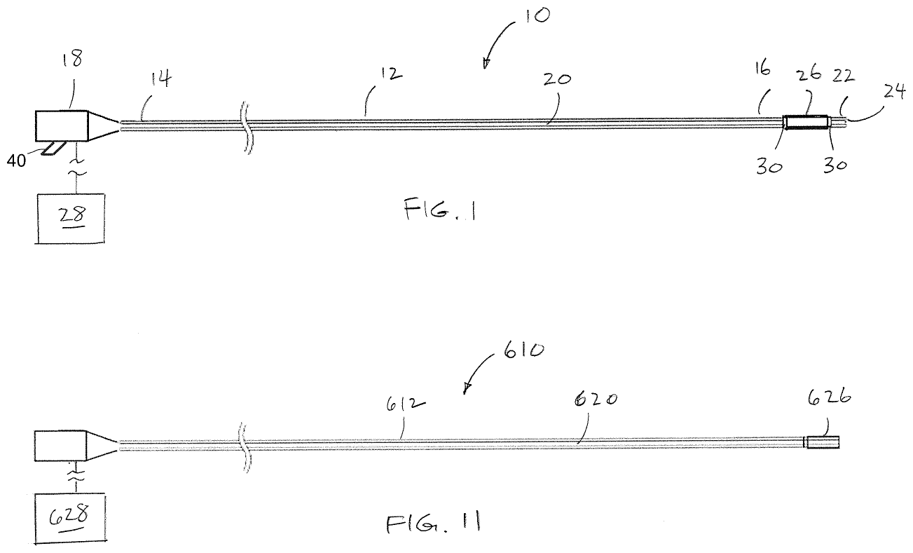

Turning to FIG. 1, an embodiment of a system 10 herein includes a flexible microcatheter 12 having a proximal end 14 and a distal end 16. The microcatheter 12 preferably has a length between two and eight feet long, and preferably has an outer diameter of between 0.67 mm and 3 mm (corresponding to catheter sizes 2 French to 12 French), and an infusion lumen 20 with an inner diameter of between 0.25-1.85 mm, depending on the intended therapeutic. For example, for relatively low viscosity liquid therapeutics, an inner diameter range of 0.254 mm-0.889 may be preferred; for relatively medium viscosity embolic agents or cells therapeutics, an inner diameter range of 0.46 mm-1.42 mm may be preferred; and for relatively higher viscosity gel plugs, an inner diameter range of 0.51 mm-1.85 mm may be preferred. The microcatheter 12 preferably includes an inner liner, an inner braid, and an outer coating. By way of example, the liner may be made of fluorinated polymer such as polytetrafluoroethylene (PTFE) or fluorinated ethylene propylene (FEP). By way of example, the braid is made of metal such as stainless steel or nickel titanium alloy, or a polymer such as polyethylene terephthalate (PET) or liquid crystal polymer or any other standard or specialty material used in making braids used in the bloodstream. By way of example, the outer coating is made of a polyether block amide thermoplastic elastomeric resin such as Pebax®, polyurethane, polyamide, copolymers of polyamide, polyester, copolymers of polyester, fluorinated polymers, such as PTFE, FEP, polyimides, polycarbonate or any other suitable material, or any other standard or specialty material used in making catheters used in the bloodstream.

The proximal end of 14 the microcatheter is preferably provided with a hub 18. The infusion lumen 20 extends from the hub 18 through to the distal end 16 of the microcatheter and exits through a distal tip 22 at a distal orifice 24. The hub 18 and infusion lumen 20 are adapted for delivery of a therapeutic agent from outside the body of a patient to a target vessel (artery or vein) in the patient. The hub 18 is also adapted to facilitate advancement of a guidewire through the infusion lumen 20. Any hub 18 suitable for at least facilitating delivery of a therapeutic into the infusion lumen can be utilized.

In accord with a preferred aspect of the system, a vascular spasm-inducing system including a probe 26 is provided at or adjacent the distal end 22 of the microcatheter 12. An actuator 28 is operably coupled to the system 10 to power and/or trigger the probe 26. Preferably, the distal tip 22 of the microcatheter 12 protrudes beyond the spasm-inducing probe 26 such that the spasm-inducing probe is proximally displaced from the distal tip.

One or more marker bands 30 are provided proximal and/or distal of the spasm-inducing probe 26. The marker bands 30 may be radio-opaque. During use of the device, the in vivo positions of the one or more marker bands 30 viewed fluoroscopically or via other imaging technique indicates the location of the spasm-inducing probe 26 relative to anatomical landmarks.

Referring to FIG. 2, in accord with one embodiment of the system, the spasm-inducing probe 26 is a radiofrequency emitter, and the actuator 28 is a sine wave current generator. The radiofrequency probe 26 includes first and second displaced electrodes 32, 34. Electrical control wires 36, 38 extend along the length of the microcatheter 12 to the first and second electrodes 32, 34. The control wires 36, 38 may be incorporated into the braid of the microcatheter, may extend along the exterior of the microcatheter, may extend within the infusion lumen, or be otherwise embedded within the lining or outer polymer coating of the microcatheter. When the actuator 28 is activated, the actuator causes a suitable radiofrequency to be emitted at the probe 26 to cause a spasm which narrows a portion of a blood vessel about the probe. In the present embodiment, the actuator 28 is set to provide insufficient energy to the probe 26 to cause ablation or preferably other permanent tissue damage.

In accord with optional aspect of the system, a secondary infusion lumen 40 accessible via the hub 18 lead to one or more openings 42 at a distal end of the probe 26. Preferably a plurality of openings 42 are circumferentially displaced at or adjacent the probe to disperse an agent radially relative to the catheter 12. The secondary infusion lumen 40 and the openings 42 are provided for delivery of a vasodilator, in accord with the method described below.

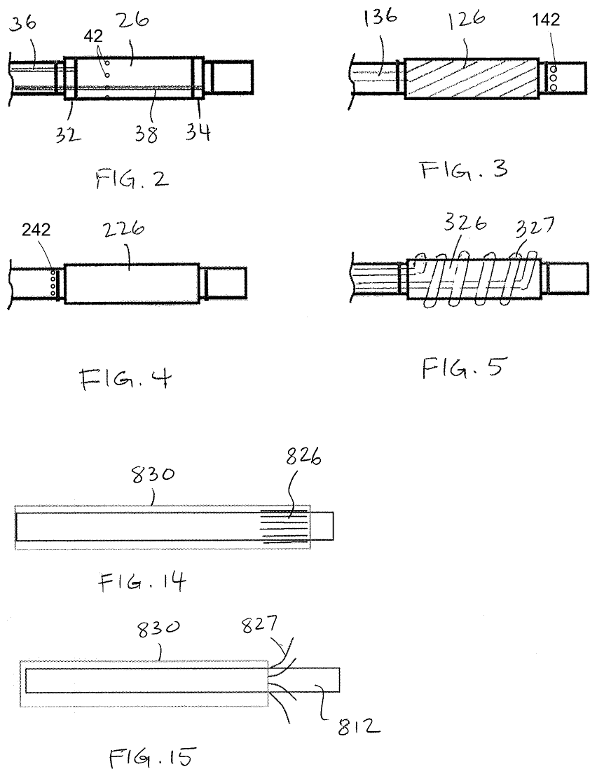

Turning to FIG. 3, in accord with another embodiment of the system, the spasm-inducing probe 126 is an ultrasound emitter. The ultrasound emitter is a high frequency sonic probe. The actuator 28 (FIG. 1) includes an ultrasound generator and power source. One or more control wires 136 extend along the length of the catheter from the actuator 28 to the probe 126. When the actuator is activated, a high frequency sonic pulse is generated and emitted at the probe, which causes vascular tissue adjacent the probe to spasm. The spasm causes the vascular tissue to narrow down against the probe. This embodiment of the system can be used in the same manner as the prior embodiment of the system. The probe 126 can also be provided in association with a secondary infusion lumen for delivery of a vasodilator as described above. By way of example, the openings 142 for the secondary infusion lumen are shown distal of the probe 126.

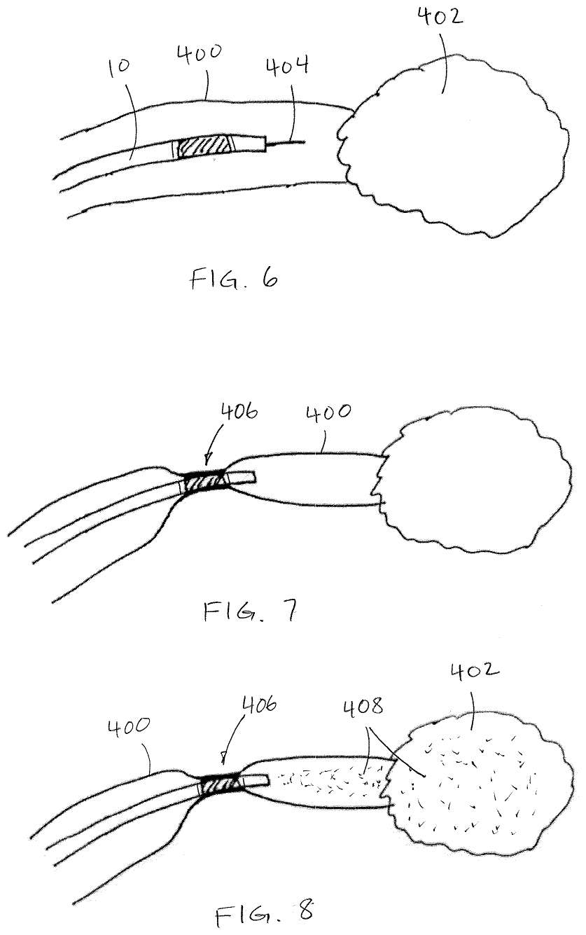

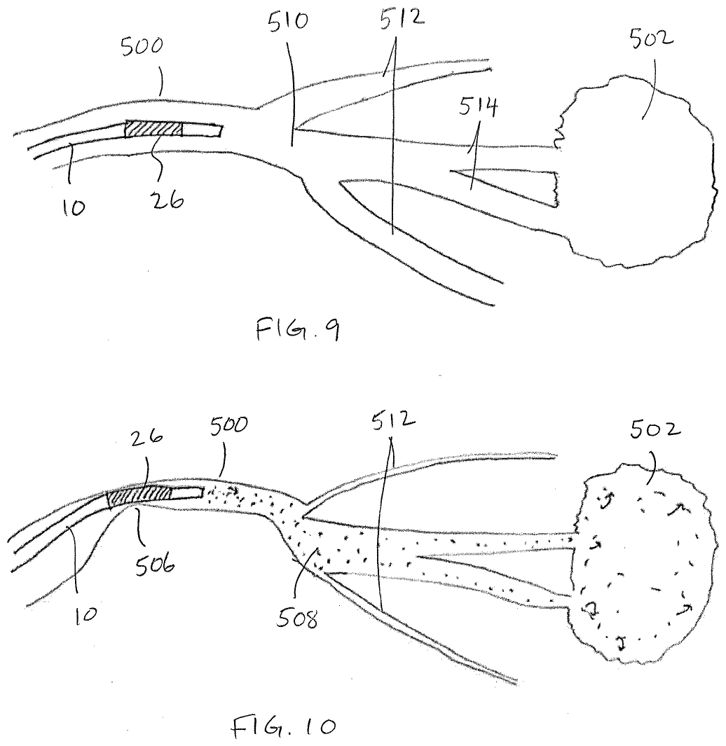

Turning now to FIG. 4, in accord with another embodiment of the system, the spasm-inducing probe is a chemical emitter 226. The chemical emitter 226 can controllably release vasoconstrictor chemicals. Such chemicals include, but are not limited to, adrenaline, oxymetazoline, phenylephrine, epinephrine, xylometazoline, naphazoline, tetryzoline, angiotensin II, vasopressin, felypressin, and midodrine. In an embodiment, the emitter 226 is preferably adapted to release the vasoconstrictor chemicals by local diffusion from an element on or attached to the catheter that slowly dissolves or is released from a compartment in the catheter. In an embodiment, the chemical emitter releases the chemical agent through circumferential side holes arranged about the circumference of the catheter. The chemical agent may be infused through a dedicated secondary lumen of the microcatheter. In such embodiment, the actuator can be a syringe or other dispenser for the chemical agent. In another embodiment, the chemical agent may be stored at the distal end of the microcatheter and covered in a membrane. The permeability of the membrane to the chemical agent may be modified by passing a current through the membrane. Control wires extend to the membrane, and the actuator includes a current source and trigger to extend current along the control wires to alter the permeability of the membrane to permit the chemical agent to be released from the element. Alternatively, a retractable cover may be provided over the chemical agent and retracted to expose the chemical agent when intended. In yet another embodiment, the chemical agent may be stored in an electroactive polymer gel on the probe. A current or voltage is applied to the gel to cause release of the chemical agent. If incorporated in a gel, the chemical agents may be released from the gel by way of (1) forced convection of the chemical out of the gel along with syneresed/expelled water due to the electric field; (2) diffusion; (3) electrophoresis of charged drugs; and (4) drug release upon erosion of electro-erodible gels. Other systems for releasing a chemical agent operable to induce spasms also can be used. This embodiment of the system can be used in the same manner as the prior embodiment of the system. The secondary lumen can be used for delivery of both the chemical vasoconstrictor and chemical vasodilator. Alternatively, separate lumen and openings 242, shown by way of example only proximal of the probe, may be provided for delivery of a vasodilator relative to the vasoconstrictor.

In accord with another embodiment of the system, the spasm-inducing probe is a cryogenic probe 326. The cryogenic probe 326 may be cooled by a cryogenic cooling fluid that is, for example, circulated through a coil 327. The cryogenic fluid can be cooled water or air, or even liquid nitrogen or other fluid, through an insulative pathway in or about the catheter, from the proximal end of the microcatheter to the coil. Alternatively, a thermoconductive band can be utilized rather than a coil. As yet another alternative, the cryogenic fluid can be injected into an expandable balloon to facilitate placement of the cooling fluid in proximity to the tissue of the vessel. Exemplar cryogenic fluids include cooled water, liquid argon or liquid nitrogen, and cooled gases. The actuator 28 may include a source of cryogenic fluid, and the trigger may initiate circulation of the cryogenic fluid to the thermoconductive band from a syringe or pump. In an embodiment, the cryogenic probe cools the local tissue of the vasculature by 5 to 10 C to produce mild to moderate vasoconstriction, 10 to 15 C to produce moderate to high vasoconstriction, and 15 to 25 C to produce maximal vasoconstriction. This embodiment of the system can be used in the same manner as the prior embodiment of the system. The probe 326 can also be provided in association with a secondary infusion lumen for delivery of a vasodilator to dilate the constricted the vessel at the end of a procedure. Alternatively, the probe 326 can be adapted to also be warmed, e.g., by fluid or electricity, to reverse the vasoconstrictive effect of cooling.

In addition, to the above exemplar spasm-inducing probes, other probe types can similarly be used. For example, the probe can be an infrared light probe. The probe can include fiber optics and elements that can focus and/or disperse the infrared energy in a manner that sufficiently heats up and irritates the vessel wall to cause vessel constriction.

Further, the probe can include a mechanical vibrator. The actuator for the vibratory element can adjust the frequency and amplitude of the vibration at the probe. By way of example, the vibratory element can be a piezoelectric or electromechanically vibrator. The vibratory element may operate in the range of 5 Hz to 20.00 kHz. More preferably, the element may operate in the 10-100 Hz range. It is expected that a lower frequency and higher amplitude vibration provides the stimulus necessary to cause spasm of the vessel. Other variations of the controls of the actuator may also provide suitable stimulus. The vibratory element probe can be provided in association with a secondary infusion lumen for delivery of a vasodilator as described above.

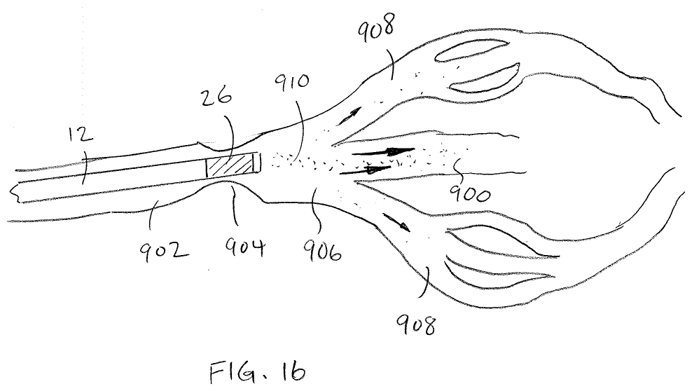

Referring to FIGS. 14 and 15, in accord with yet another embodiment of the system, the spasm inducing probe 826 is a mechanical irritation device. The probe 826 includes bristles 827 at the end of the catheter 812. The bristles 827 are deployable from a retractable sheath 830 (FIG. 14) or otherwise adapted to be covered until ready for use whereupon the sheath 830 is retracted (FIG. 15) by operation of an actuator, such as at a handle to expose the bristles 827. The bristles 827 may be a fine elastic construct and made from, for example, nickel titanium or plastic. The bristles 827 should be very soft to prevent damage to the tissue. The bristles 827 are short so that their ends are adapted to contact and scrape against the vessel wall in an irritating, tickling action. The bristles may also be coupled to a vibration element (discussed above) to amplify the irritating effect on the tissue of the vasculature and resultant spasm. The probe 826 can also be provided in association with a secondary infusion lumen for delivery of a vasodilator as described above.

The system 10 can be used to inject a therapy into a target vessel branching from a larger primary vessel and communicating with, for example, a solid tumor of an organ. In some cases, the tumor can be a cancerous tumor, such as a tumor specific to, for example, cancer of the pancreas, spleen, or small intestines. In addition, other non-cancerous diseased states of organs can also be treated using the systems and methods.

As described below, the treatment system is used to provide the therapy to the tumor within a target region of the organ, to enable targeted treatment of the targeted region by the therapy, and substantial isolation of the therapy within the target region, all without isolating a larger region than necessary from blood flow during the treatment procedure. This is in contrast from treatment that are provided in systemic circulation through the body.

Turning now to FIGS. 1 and 6, the distal end of the system is tracked to a location of interest; for example, a small vessel 400 feeding a tumor 402. To facilitate the tracking, a guidewire 404 may be advanced to the vessel 400 of interest. In addition, the intravascular tracking of the microcatheter system 10 into the vessel 400, whether over a guidewire 404 or independent of the guidewire, is preferably performed under imaging. The positioning of the spasm-inducing probe 26 (in which probe 26 is intended to reference any of the embodiments of the spasm-inducing probe) at the intended location within the vascular anatomy can be confirmed by infusing a contrast agent through the infusion lumen 20 and out of the distal orifice 24 of the microcatheter to visualize the vascular anatomy and the location of the marker band(s) 30 relative to the illuminated anatomy.

Referring to FIGS. 1 and 7, once the treatment system 10 is confirmed to be in position in the target vessel 400, the actuator 28 is activated to cause the spasm-inducing probe 26 to induce spasm 406 in the vessel. The spasm causes the vessel to narrow down against the system at the spasm inducing probe 26.

Then, referring to FIGS. 1 and 8, while the vessel 400 is under spasm 406, a therapeutic agent 408 is infused through the hub 18, through the infusion lumen 20 and out of the distal orifice 24 under pressure into the tumor 402 or other target tissue. The narrowed vessel at the spasm 406 prevents reflux of the therapeutic agent 408 and maintains high pressure of the therapeutic delivery downstream of the spasm 406 into the tumor or other target tissue.

Once the therapeutic agent has been delivered, before removing the catheter a therapeutic action can be taken to at least partially reverse the constriction on the vessel. As discussed above, a chemical vasodilator can be delivered through a secondary infusion lumen. Alternatively, where the constriction was caused by cooling, the tissue can be warmed. Any other suitable method can be used to alleviate the constrict or dilate the vessel. Then, the treatment system can be withdrawn from the patient.

Turning now to FIGS. 1, 9 and 10, in accord with another method, applicable to any of the systems described above, the system 10 can be used to control flow within the vasculature before and during delivery of a therapeutic agent in order to (re)direct and optimize flow of the therapeutic agent to a tumor 502. The distal end 16 of the microcatheter 12 of the system 10 is tracked to a target vessel 500 of interest. This may be performed using a guidewire, contrast agent, and/or imaging, as described above. The tracked location is preferably upstream (proximal) of a branch system 510 leading to both healthy vasculature 512 and tumor vasculature 514. Once the spasm-inducing probe 26 of the treatment system 10 is confirmed to be in position in the target vessel 500, the actuator 28 is activated to cause the spasm-inducing probe 26 to induce a spasm 506 in the vessel 500. With spasm induced, flow to and fluid pressure in both the healthy vessels 512 and tumor vessels 514 is reduced. The healthy vessels 512 respond to the reduced flow and pressure by constricting. However, due to the disease in the tumor vasculature 514, the tumor vasculature will fail to similarly constrict upon similarly being subject to lower fluid pressure; the tumor vasculature will either fail to constrict or constrict to a far lower extent.

Then, a therapeutic agent 508 is infused through the infusion lumen of the system, while the spasm-inducing probe 26 remains activated. The vasoconstriction of the healthy branches 512 causes the flow of the therapeutic agent 508 to be primarily directed through the tumor vasculature 514, which remains substantially open and substantially unconstricted, and into the tumor 502. Further, the vascular volume available downstream of the spasm also allows the infusion into the tumor to be at a higher pressure.

At the end of infusion, the pressure in the vessels is high, and the healthy tissue will attempt to vasodilate to reduce pressure. As the tumor vasculature is unable to similarly adapt, this would redirect flow toward healthy tissue. As this is counter to the purpose of maximizing flow toward diseased tissue, the spasm-inducing probe 26 preferably remains active, reducing antegrade flow in the vessels even at the end of the infusion to limit healthy vessel dilation. With the healthy vessels restrained from dilation, the therapy is provided the best route through tumor vasculature to the tumor.

In accord with one aspect of the method, the spasm-inducing probe may be actuated and the therapy may be infused in pulses to maximize the constriction of the healthy vessels. The pulses may be spaced apart by 0.3 to 60 seconds. The pulses may optimize the constriction and prevent dilation of the healthy vasculature for therapeutic redirection and aid in uptake of the agent at the tumor.

At the end of the therapeutic procedure, optionally before removing the treatment system from the patient, a therapeutic action can be taken to at least partially reverse the constriction on the vessel. As discussed above, a chemical vasodilator can be delivered through a secondary infusion lumen. Alternatively, where the constriction was caused by cooling, the tissue can be warmed. Any other suitable method can be used to alleviate the constriction or otherwise dilate the vessel. The treatment system is then withdrawn from the patient.

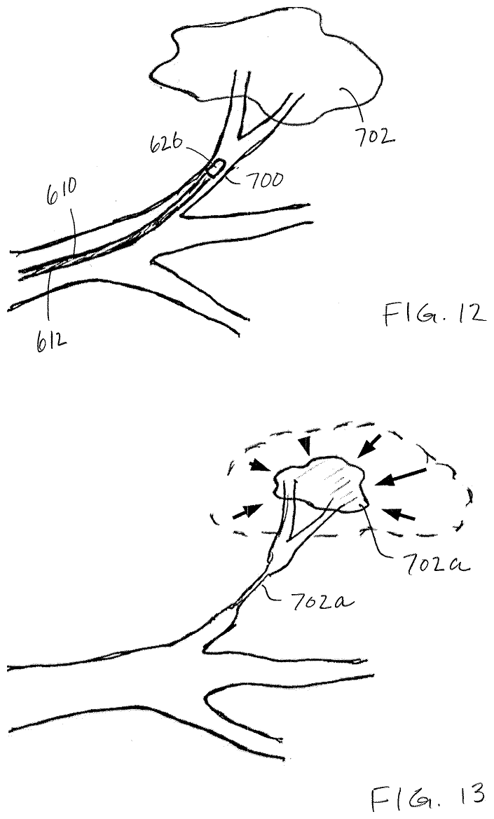

Turning now to FIG. 11, another system 610 is shown. The system 610 is substantially similar to system 10 described above (with like parts having reference numerals incremented by 600). In distinction from the spasm-inducing probe 26, the system 10 includes an ablation probe 626. Ablation probe and spasm-inducing probe may be substantially structurally similar, different only in the energy adapted to be output at the probe. That is, while spasm-inducing probe 26 is adapted to cause vascular tissue to temporarily spasm and not intentionally cause permanent occlusion of a vessel, ablation probe 626 is adapted to release sufficient energy to result in a more long-term change, and even a permanent change, in the vascular tissue so as to result in occlusion thereof or otherwise prevent vascular nourishment of the organ or tissue fed by the vessel. As such, the actuator 628 may be adapted to causes a different radiofrequency energy (e.g., from a higher current), a different ultrasound energy (e.g., from a different frequency and/or amplitude), a different chemical energy (e.g., from different chemicals and/or different amounts of chemicals); or different thermal energy (e.g., from different cooling temperature using the same or different cryogenic fluids, or an elevated temperature). In addition, other ways of achieving ablation can be used. For example, a microwave emitter can be provided at the ablation probe 626. The actuator 628 can power and trigger the ablation emitter. By way of example, the actuator is adapted to operate the probe 626 at 5.80 GHz at 50 W; although it can be operated at other suitable bands and powers. The microcatheter 612 is not necessarily required to infuse a therapeutic agent, as the intended therapeutic effect is provided by ablation of tissue, as described below. However, the microcatheter 612 preferably includes a central lumen 620 sized for advancing the microcatheter over a guidewire.

Referring to FIGS. 12 and 13, in use, a mapping procedure is performed to identify the significant arterial vessels feeding a solid tumor. Once a target vessel 700 is identified, the femoral or radial artery is accessed, and a guidewire is advanced through the artery to the target vessel feeding the tumor. The microcatheter 612 is then advanced over the guidewire to the target vessel 700. Then, the guidewire is removed. Alternatively, the microcatheter is advanced directly, without a guidewire. The ablation probe 626 is then activated to ablate the tumor-feeding arterial vessel 700. In one embodiment, the ablation probe is initially activated to first cause spasm and then subsequently activated to cause ablation. This may be effected by activating the probe 626 at an initial lower energy, or to initially provide a small chemical release, etc., and subsequently operated to cause ablation. Ablation results in occlusion and/or collapse of the artery 700a, preventing nourishing blood flow to the tumor 702. This will prevent further growth of the tumor and/or reduce the size of the tumor 702a. The system can be repositioned for ablation in additional vessels or removed.

The systems described above may be used to control internal bleeding. It is noted that physicians may use embolics to stop internal bleeding in, without limitation, the lungs, the spleen, and the pancreas. The above described systems, in any of the vasospasm or vasoablative embodiments, by operation to cause constriction, can be used to immediately slow or stop blood loss as soon as the device is placed and activated. Then, a secondary therapy can be infused locally into the patient. The secondary therapy may include a gel-foam, glue or liquid embolics, embolic coils, or embolic beads. In accord therewith, turning to FIG. 16, a bleeding vessel 900 is identified. The system is advanced to a target vessel 902 upstream of the bleeding vessel 900. The system is operated to generate vasoconstriction at 904. The vasoconstriction 904 causes a pressure drop in the local system at 906 between the constriction 904 and the bleeding vessel 900. The normal vessels 908 have higher resistance than the bleeding vessel 900. Therefore, when the secondary therapy 910 is infused, the therapy 910 flows favorably to the site of damage 900 over the higher resistance normal vessels 908.

In any of the methods, the target vessel extends into or near a tumor or other diseased tissue. The target vessel may feed or drain from any of various organs, including, but not limited to, the pancreas, spleen, gastrointestinal tract, liver, lung, uterus, prostate or brain, as well as target vessels communicating with head and neck tumors. The target vessels may also be in communication with other organs or tissues of interest for treatment in other parts of the body. In embodiments, the treatment system may be introduced into or adjacent the target vessel non-endovascularly.

There have been described and illustrated herein embodiments of systems and methods for therapeutic delivery, and in embodiment pressure-enabled therapeutic delivery. While particular embodiments of the invention have been described, it is not intended that the invention be limited thereto, as it is intended that the invention be as broad in scope as the art will allow and that the specification be read likewise. Thus, while the systems and methods are primarily adapted for therapeutic treatment of humans, it has been demonstrated on porcine tissues and organs, and can be used for the treatment of mammals, in general. Both humans and animals shall be considered ‘patients’ for purpose of this disclosure. Also, the therapy delivered herein can be a single therapeutic agent, or a combination of therapeutic agents. It will therefore be appreciated by those skilled in the art that, yet other modifications could be made to the provided invention without deviating from its scope as claimed.

What is claimed is:

1. A therapeutic system for treatment in a vessel of a patient, comprising:
a) a catheter having a proximal end and a distal end with a distal tip, a first lumen defined from the proximal end and extending to the distal tip and opening at a distal orifice passing through the distal tip; and
b) a spasm-inducing system including an activatable probe at the distal end of the catheter, which when activated is adapted to induce a narrowing spasm in the vessel.

2. The therapeutic system of claim 1, wherein the distal tip protrudes beyond the probe.

3. The therapeutic system of claim 1, further comprising at least one marker provided in association with the emitter to identify the location of the probe under imaging.

4. The therapeutic system of claim 3, wherein the at least one marker is radioopaque.

5. The therapeutic system of claim 1, further comprising a hub at the proximal end of the catheter.

6. The therapeutic system of claim 1, wherein the spasm-inducing system is adapted to emit radiofrequency (RF) energy at the probe.

7. The therapeutic system of claim 1, wherein the probe of the spasm-inducing system is adapted to emit ultrasound.

8. The therapeutic system of claim 1, wherein the probe of the spasm-inducing system is adapted to emit chemicals.

9. The therapeutic system of claim 1, wherein the probe of the spasm-inducing system is a cryogenic element.

10. The therapeutic system of claim 1, wherein the probe of the spasm-inducing system is a vibratory element.

11. The therapeutic system of claim 1, wherein the probe of the spasm-inducing system is an infrared light emitting element.

12. The therapeutic system of claim 1, wherein the probe of the spasm-inducing system includes bristles adapted to irritate the vessel.

13. The therapeutic system of claim 1, wherein the catheter defines a second lumen opening at or adjacent the probe.

14. The therapeutic system of claim 13, wherein the second lumen opens to disperse an infusate radially relative to the catheter.

15. A therapeutic system for treatment in a vessel of a patient, comprising:
a) a catheter having a proximal end and a distal end with a distal tip, a first lumen defined from the proximal end and extending to the distal tip and opening at a distal orifice passing through the distal tip; and
b) a probe at the distal end of the catheter; and
c) an actuator for activating the probe, whereupon activation of the probe, the probe is adapted to cause a physical change in the vessel.

16. The therapeutic system of claim 15, wherein activation of the probe is adapted to cause a narrowing spasm in the vessel.

17. The therapeutic system of claim 15, wherein activation of the probe is adapted to cause ablation of the vessel.

18. The therapeutic system of claim 15, wherein the probe is adapted to emit radiofrequency (RF) energy.

19. The therapeutic system of claim 15, wherein the probe is adapted to emit ultrasound.

20. The therapeutic system of claim 15, wherein the probe is adapted to emit chemicals.

21. The therapeutic system of claim 15, wherein the probe is a cryogenic element.

22. The therapeutic system of claim 15, wherein the probe is adapted to emit microwave energy.

23. The therapeutic system of claim 15, wherein the probe is adapted to emit thermal energy.

24. The therapeutic system of claim 15, wherein the probe of the spasm-inducing system is a vibratory element.

25. The therapeutic system of claim 15, wherein the probe of the spasm-inducing system is an infrared light emitting element.

26. The therapeutic system of claim 15, wherein the probe of the spasm-inducing system includes bristles adapted to irritate the vessel.

27. The therapeutic system of claim 15, wherein the catheter is provided with a second lumen opening at or adjacent the probe.

28. The therapeutic system of claim 27, wherein the second lumen opens to disperse an infusate radially relative to the catheter.

29. A method of therapeutic treatment comprising:
a) providing a catheter having a proximal end and a distal end with a distal tip, and a lumen extending from the proximal end to the distal end and opening at a distal orifice passing through the distal tip, the catheter provided with an actuatable probe at the distal end of the catheter;
b) advancing the distal end of the catheter into a vessel;
c) actuating the probe to cause the vessel to narrow about the probe; and
d) infusing a therapeutic agent through the lumen and out of the distal orifice into the vessel while the vessel is narrowed about the probe to a location distal of the narrowing in the vessel.

30. The method of claim 29, wherein the therapeutic agent is infused from the vessel toward a tumor.

31. The method of claim 29, wherein between the distal end of the catheter and the tumor, the vessel branches to healthy vasculature and tumor vasculature, and after activating the probe and before infusing the therapeutic agent, causing the healthy vasculature to narrow more than the tumor vasculature.

32. The method of claim 29, wherein the infusing comprising multiple infusions spaced apart in time.

33. The method of claim 29, wherein the therapeutic treatment is to treat internal bleeding.

34. The method of claim 33, wherein the therapeutic agent is one of a gelfoam, a glue embolic, a liquid embolic, an embolic coil, and embolic beads.

35. The method of claim 29, wherein the narrowed vessel constricts into contact with the probe.

36. The method of claim 29, further comprising:
after infusing the therapeutic agent, dilating the narrowed vessel.

37. The method of claim 36, wherein the dilating includes introducing a vasodilator to the narrowed vessel.

38. A method of therapeutic treatment, comprising:
a) providing a catheter having a proximal end and a distal end with a distal tip, and a lumen extending from the proximal end to the distal end and opening at a distal orifice passing through the distal tip, the catheter provided with an actuatable probe at the distal end of the catheter;
b) advancing the distal end of the catheter into an artery feeding a tumor; and
c) actuating the probe to cause ablation of the artery feeding the tumor.
